(19) United States
(12) Patent Application Publication
(10) Pub. No.: US 2022/0304608 A1
(43) Pub. Date: Sep. 29, 2022

(54) MULTIFUNCTIONAL ELECTROPHYSIOLOGICAL MONITORING SYSTEM

(71) Applicant: Umana Medical Technologies Ltd., Luqa (MT)

(72) Inventors: Adrian Attard Trevisan, Rabat (MT); ANTOINE ZERAFA, Zejtun (MT); BRENDON SULTANA, Kalkara (MT); CHRISTOPHER CAUCHI, Attard (MT); EMAN CAUCHI, Gudja (MT); ALESSANDRO GALLOPPINI, Tarxien (MT); ANDREA CAMILLERI, Zebbug (MT)

(21) Appl. No.: 17/213,119

(22) Filed: Mar. 25, 2021

(51) Int. Cl. A61B 5/251; A61B 5/0205; A61B 5/28; A61B 5/291; A61B 5/296; A61B 5/00; A61B 5/274

(52) U.S. Cl. CPC A61B 5/251; A61B 5/02055; A61B 5/28; A61B 5/291; A61B 5/296; A61B 5/0006; A61B 5/274; A61B 5/02416

(57) ABSTRACT

A multifunctional electrophysiological monitoring system includes a body-worn tattoo sensor pattern and an electrophysiological monitor connected to sensor contacts of the pattern. A dongle performs a supplemental function, such as acquiring information about a physiological parameter. The dongle interfaces with a port of the monitor. The monitor interfaces wirelessly with a mobile communication device and a monitor network. In an embodiment, the monitor transmits ECG, EMG, or EEG signals. The dongle may provide further monitoring such as body temperature or blood oxygen saturation level. In some embodiments a plurality of dongles may be connected to the monitor.

### CROSS REFERENCE TO RELATED APPLICATION

None

### TECHNICAL FIELD

The present invention pertains generally to electrophysiological monitoring, and more particularly to temporary tattoo biosensors and multifunctional health monitoring systems in cooperation therewith.

### BACKGROUND OF THE INVENTION

Conventional electrophysiological monitoring methods, such as electrocardiography (ECG or EKG), electromyography (EMG), and electroencephalography (EEG), use conductive electrodes adhered to the skin of a patient with an adhesive or electrolytic gel. The electrodes are wired to a data acquisition unit. The size and wired connections of such systems make them impractical and inconvenient for long-term or mobile use. In addition, gels have a short useful lifetime before drying out. Furthermore, adhesives, gels, and the preparation method used to attach gelled sensors often irritate the patient's skin.

Recently, conductive polymer films have been demonstrated to have properties suitable for use as biosensors. Conformable tattoo biosensors having submicrometric thickness were demonstrated in polymer films. Other tattoo biosensors have been demonstrated using silver nanoparticles, polymer-enhanced carbon, and graphene. Demonstrated applications include ECG, EMG, and EEG monitoring.

Many smaller biosensors have focused on providing a single sensing functionality in an effort to reduce overall size of the sensor system. There exists a need for a electrophysiological monitoring system that is multifunctional, may be discretely and comfortably worn, and may be customized to a desired patient function.

### BRIEF SUMMARY OF THE EMBODIMENTS

Embodiments disclosed herein are directed to a electrophysiological monitoring system providing multiple functionalities through the connection of one or more dongles. The system includes a tattoo sensor device and a monitor which is monitors and processes ECG, EMG, or EEG signals. Through the connected dongle, additional functionalities may be provided. The system is suitable for long-time monitoring of a patient's vital parameters in ambulatory, in-home, or outpatient settings.

The embodiments disclosed herein may be summarized as follows.

### Embodiment 1

A multifunctional electrophysiological monitoring system, including:

a conductive polymer pattern (102) configured for transfer to the skin of a patient, the pattern including a plurality of sensor regions (110) each connected to a patterned lead (120) having a terminus (130) adjacent to a common contact region (140);

a plurality of sensor contacts (150) arranged within the contact region, each terminus of the patterned leads in electrical communication with one of the sensor contacts;

an electrophysiological monitor (200) having:
a housing (202) having a rear face (210) and a sidewall (210);
a plurality of monitor contacts (230) on the rear face, each of the monitor contacts configured to directly connect to one of the sensor contacts;
an integrated circuit (240) configured to digitize a first biosignal;
a memory (260);
a transceiver (270) configured for wireless transmission of the digitized signals; and
an electrical connection port (206) in the sidewall;

a dongle (600) configured to perform a supplemental function and having a connector (602) configured to interface with the electrical connection port to communicate the information to the monitor; and

a mobile communication device (300) having a transceiver (370) configured for wireless communication with the monitor and a processor (310) configured to process the digitized signals.

### Embodiment 2

The system of Embodiment 1, wherein the supplemental function includes, acquiring information about a physiological parameter, the information different from the first biosignal sensed by the monitor.

### Embodiment 3

The system of Embodiment 1 or 2, wherein the first biosignal is selected from the group consisting of: an ECG signal, an EEG signal or an EMG signal.

### Embodiment 4

The system of Embodiment 2 or 3, wherein the information acquired by the dongle is selected from the group consisting of: a temperature signal, a blood oxygen saturation signal, an analyte concentration, a pH measurement, a bioimpedance measurement, or a photoplethysmogram signal.

### Embodiment 5

The system of any one of Embodiments 1 to 4, wherein the monitor includes a plurality of electrical connection ports in the sidewall.

### Embodiment 6

The system of Embodiment 5, further including an additional dongle configured to acquire information regarding an additional biosignal and having a connector configured to interface with one of the electrical connection ports to communicate the information to the monitor.

### Embodiment 7

The system of Embodiment 6, wherein the additional biosignal relates to a different biological property than the first biosignal.

### Embodiment 8

The system of any one of Embodiments 1 to 7, wherein the connector of the dongle is configured to interface with the electrical connection port to receive power.

### Embodiment 9

The system of any one of Embodiments 1 to 8, wherein the dongle is sized to be supported by the monitor through the interface of the connector with the electrical connection port.

### Embodiment 10

The system of any one of Embodiments 1 to 9, wherein the dongle has a dongle thickness of less than a sidewall thickness of the monitor.

### Embodiment 11

The system of any one of Embodiments 1 to 10, wherein each of the monitor contacts is configured to directly connect both mechanically and electrically to one of the sensor contacts.

### Embodiment 12

The system of any one of Embodiments 1 to 11, wherein the dongle includes a microcontroller (610) electrically coupled to the connector and a transducer (608) electrically coupled to the microcontroller.

### Embodiment 13

The system of any one of Embodiments 1 to 12, wherein the dongle includes a transducer electrically coupled to a dongle sensor contact (604) configured to contact the skin of the patient.

### Embodiment 14

The system of any one of Embodiments 1 to 13, wherein the supplemental function of the dongle includes acquiring information about a physiological parameter through electrical communication with an electrode.

### Embodiment 15

The system of any one of Embodiments 1 to 14, wherein the dongle includes a sensing module to acquire information about a physiological parameter.

### Embodiment 16

The system of any one of Embodiments 1 to 15, wherein the dongle includes a transceiver configured for wireless communication with the mobile communication device

### Embodiment 17

The system of any one of Embodiments 1 to 16, wherein each dongle can be connected mechanically and electrically to one or multiple dongles.

These and other aspects of the embodiments will be better appreciated and understood when considered in conjunction with the following description and the accompanying drawings. The following description, while indicating various embodiments and details thereof, is given by way of illustration and not of limitation. Many substitutions, modifications, additions, or rearrangements may be made within the scope of the embodiments, and the embodiments may include all such substitutions, modifications, additions, or rearrangements.

### BRIEF DESCRIPTION OF THE DRAWINGS

Non-limiting and non-exhaustive embodiments of the multifunctional electrophysiological monitoring system are described with reference to the following figures, wherein like reference numerals refer to like parts throughout the various views unless otherwise specified.

FIG. 1 is an example illustration of an embodiment of an electrophysiological monitoring system in use.

FIG. 2 is an exploded illustration of an embodiment of the electrophysiological monitoring system.

FIG. 3A is a top plan view of an embodiment of a wearable portion of the system; and FIG. 3B is a top plan view of the embodiment with sensor contacts.

FIG. 4A is a top plan view of another embodiment of a wearable portion; and FIG. 4B is a perspective view of the wearable portion and a monitor of the system.

FIGS. 5A-5C are front, rear, and perspective views, respectively, of a monitor of the system.

FIGS. 6A-6B are front and rear perspective views, respectively, of a dongle of the system.

FIG. 7 is a schematic representation of an embodiment of the electrophysiological monitoring system.

FIG. 8 is an enlarged cross-sectional view along the line VIII-VIII of FIG. 3.

FIGS. 9A-9C are schematic representations of embodiments of the dongle.

Skilled artisans will appreciate that elements in the figures are illustrated for simplicity and clarity and have not necessarily been drawn to scale. For example, the dimensions of some of the elements in the figures may be exaggerated relative to other elements to help improve understanding of various embodiments. Also, common but well-understood elements that are useful or necessary in a commercially feasible embodiment are often not depicted in order to facilitate a less obstructed view of these various embodiments.

### LIST OF DRAWING REFERENCE NUMERALS

100 wearable portion

102 conductive polymer pattern

104 substrate sheet

104a front liner

104b film

104c adhesive layer

110 sensor region

120 patterned lead

130 terminus

140 contact region

150 sensor contact

200 electrophysiological monitor

202 housing

204 sidewall

206 electrical connection port

208 charging port

210 rear face

230 monitor contact

240 integrated circuit

250 motion sensor

260 memory

270 transceiver

300 mobile communication device

310 processor

320 display

370 transceiver

380 battery

400 monitor network

410 cloud

420 user client

430 medical professional client

440 other client

500 electrophysiological monitoring system

600 dongle

602 connector

604 dongle sensor contact

606 contactless sensor

608 transducer

610 microcontroller

### DETAILED DESCRIPTION OF THE INVENTION

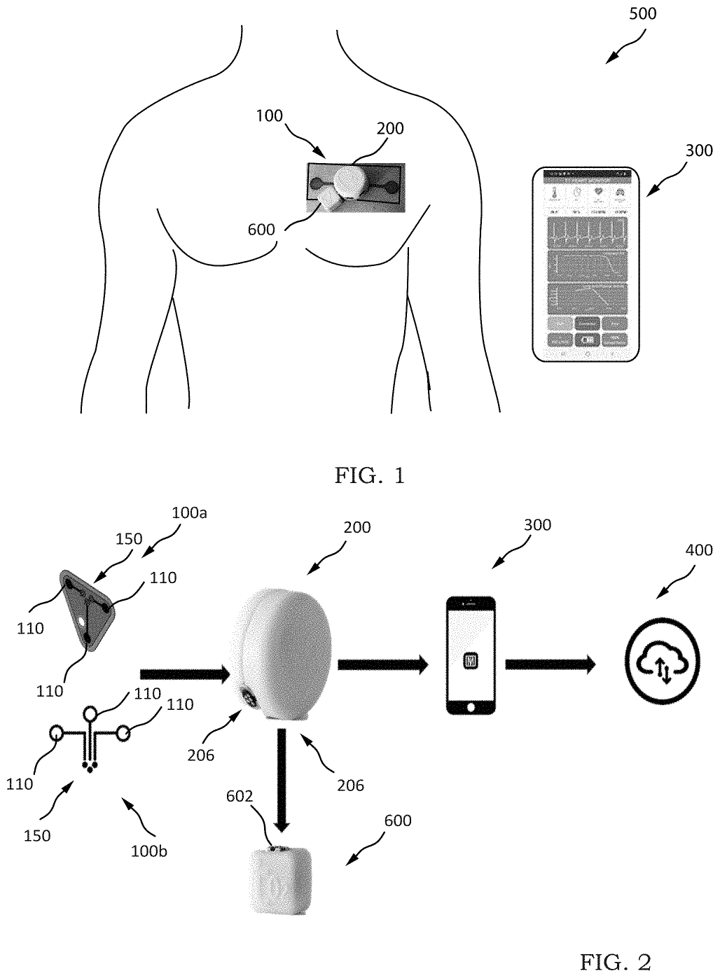

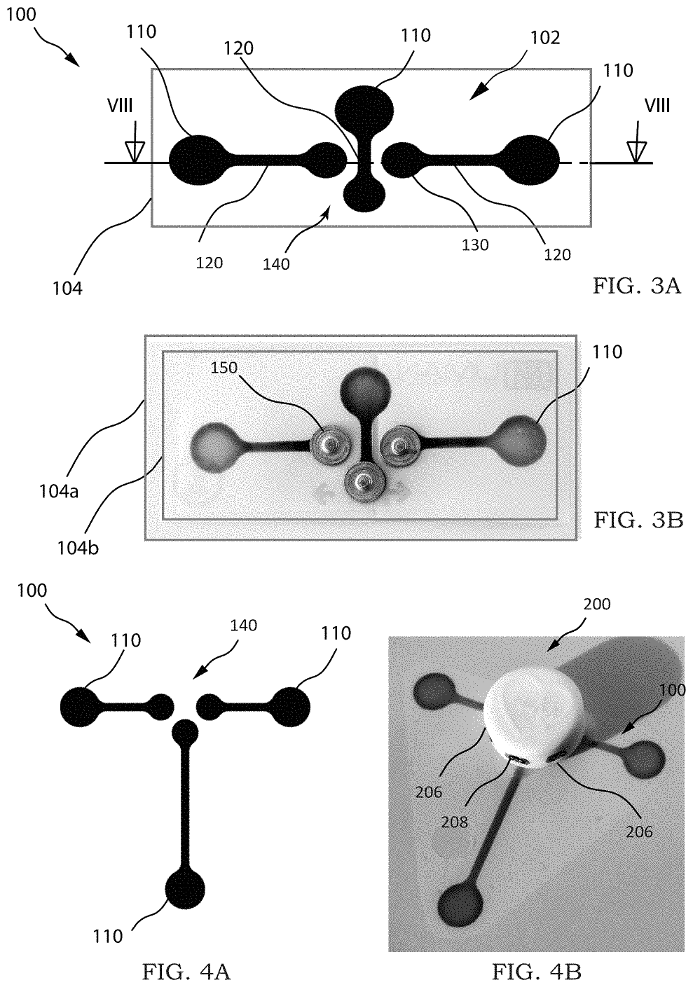

FIG. 1 is an example illustration of an embodiment of a multifunctional electrophysiological monitoring system 500 being worn by a patient, the wearable portion of the system generally designated as 100. FIG. 2 is an exploded illustration the system, FIGS. 3A-3B and 4A are top plan views of a wearable portion of the system, and FIG. 4B shows a monitor 200 connected to wearable portion 100. Wearable portion 100 includes a conductive polymer pattern 102 on a substrate sheet 104, such as decal transfer paper which is commonly used for temporary tattoos. Conductive polymer pattern 102 includes a plurality of sensor regions 110 (hereinafter referred to as sensors), three sensors 110 being present in the embodiments shown in FIGS. 1-4. In other embodiments, 2, 4, 5, 6, or another number of sensors 110 may be included.

FIG. 1 shows conductive polymer pattern 102 being worn by a patient, in a manner suitable for electrocardiography (ECG or EKG). Conductive polymer pattern 102 is transferred to the skin of a patient in the manner of applying a temporary tattoo. Each sensor 110 of pattern 102 is connected to a patterned lead 120; in an exemplary embodiment patterned lead 120 may be formed of the same conductive polymer as sensor 110. Each patterned lead 120 has a terminus 130 adjacent to or located within a common contact region 140. Each terminus 130 is in electrical connection with a sensor contact 150 located within contact region 140. Three of sensor contact 150 are present in FIG. 2 embodiments of wearable portion 100a, 100b.

Electrophysiological monitor 200 is configured to directly connect with sensor contacts 150 located within contact region 140. In FIG. 2 either wearable portion 100a or 100b may be used with monitor 200 in a particular embodiment of the system. In some embodiments, wearable portion 100 may be worn near a skeletal muscle (such as locations on an arm or leg) for use in electromyography (EMG). In some embodiments, wearable portion 100 may be worn on the scalp for use in electroencephalography (EEG).

Monitor 200 includes an integrated circuit configured to digitize a first biosignal, such as an electrophysiological signal. A dongle 600 connects to monitor 200 and acquires information regarding a second biosignal. In embodiments, monitor 200 is configured to connect to a plurality of dongles 600, each of which may acquire a different biosignal. In embodiments, the second biosignal relates to a different biological property than the first biosignal. In this way the system is considered to be multifunctional, and by exchanging one dongle for another dongle with different functionality, the sensing capabilities of the system may be readily changed. Monitor 200 is in wireless communication with a mobile communication device (MCD) 300. MCD 300 is in communication with a monitor network 400, which may be a cloud network or other internet network.

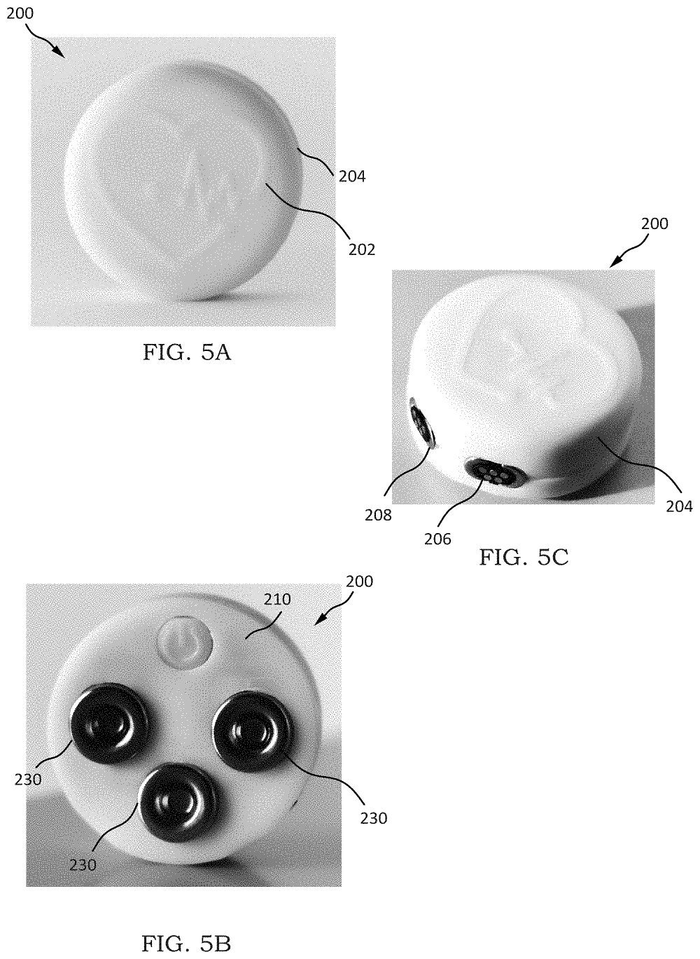

FIGS. 5A-5C are front, rear, and perspective views, respectively, of monitor 200. Monitor 200 includes a housing 202 having a rear face 210 and a sidewall 204. Sidewall 204 has one or more electrical connection ports 206 each configured to interface with a dongle 600. Two electrical connection ports 206 are shown in the embodiment of FIG. 5C. In other embodiments, one, three, four, five, or another number of electrical connection ports 206 may be present. Sidewall 204 may have other ports, such as a charging port 208 (see FIG. 4B) for recharging a battery of the monitor.

A plurality of electrical monitor contacts 230 are located on the rear face 210 of the monitor (three contacts 230 are shown in FIG. 5B). Contacts 230 are located so that when monitor 200 is positioned over contact region 140 with rear face 210 facing the skin, one of monitor contacts 230 is in electrical connection with each sensor contact 150, which in turn is in electrical connection with one of the patterned leads 120 of the conductive polymer pattern 102 (refer also to FIG. 2). For example, monitor 200 may have three monitor contacts 230. When used with a tattoo sensor having three of sensor 110, each monitor contact is in electrical connection with one sensor contact 150. When the same example monitor is used with a tattoo sensor having two of sensor 110, one monitor contact 230 is in electrical connection with each electrically connected sensor contact 150, while the remaining monitor contact 230 is unused (not connected).

Contact region 140 is configured such that a monitor having electrical contacts for direct electrical connection with some or all of sensor contacts 150 may be easily and conveniently positioned over contact region 140 (see FIGS. 4A-4B). This direct electrical connection is made without the use of wires or leads. In some embodiments, monitor contacts 230 connect both mechanically and electrically to sensor contacts 150. In embodiments, sensor contacts 150 are configured for snap-fitting to the monitor, for example sensor contacts 150 may be a male or female electroconductive stud configured for snap-fitting to the complementary female or male monitor contact 230.

Monitor contacts 230 may be electroconductive studs having an overall diameter of about 34 mm. Monitor 200 or contact region 140 may have indicia marking proper placement of monitor 200 over contact region 140. In an embodiment, monitor 200 has a major dimension of about 34 mm and a thickness of 13 mm.

In an embodiment each sensor region 110 has a diameter of about 15 mm. In embodiments the spacing between sensors 110 is between about 50 mm and about 80 mm. In embodiments, patterned leads 120 have a width on the order of 2.5 mm. In other embodiments, patterned leads may be wider to support a longer length of lead.

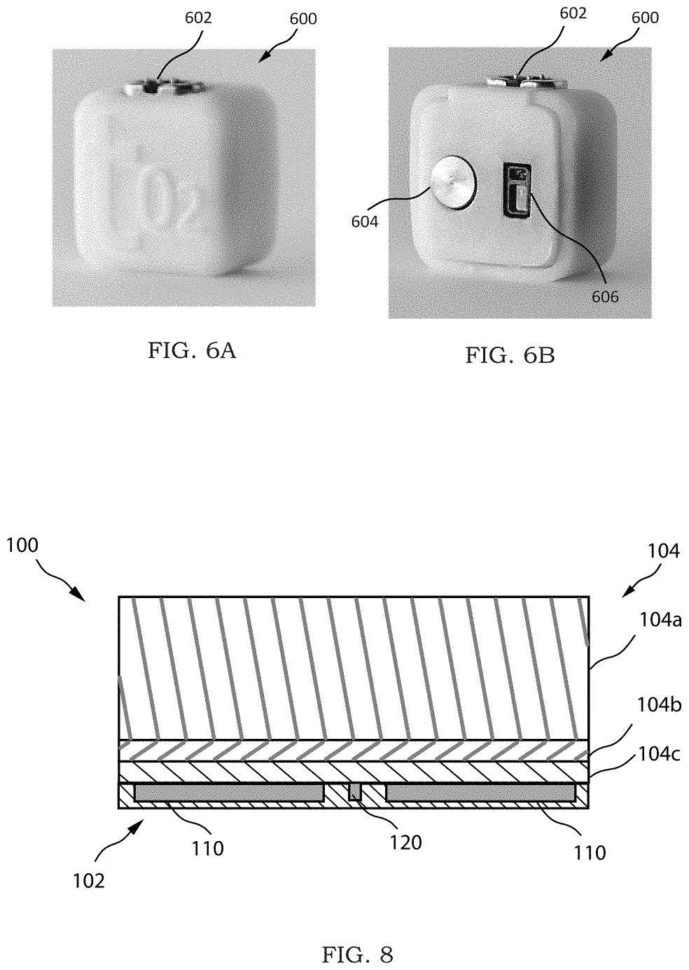

FIGS. 6A-6B are front and rear perspective views, respectively, of dongle 600. Dongle 600 has a connector 602 configured to interface with electrical connection port 206 of the monitor. The interface between connector 602 and electrical connection port 206 may be, for example, a plug with one or more pins mating to a jack or receptacle with one or more sockets configured to receive the pins. The interface may have magnetic features. The interface may be keyed such that the components will only mate in one position. The interface may include a locking or unlocking mechanism or a quick release mechanism.

Dongle 600 may be configured to acquire information regarding a second biosignal and communicate the information to monitor 200. Dongle 600 may include a dongle sensor contact 604, as shown in FIG. 6B on a rear, or skin-facing, side of the dongle. More than one dongle sensor contact 604 may be present in a given dongle. In one embodiment, sensor contact 604 is a temperature conductive stainless-steel stud. A contactless sensor 606 may also be included, such as a reflective-type SpO2 module. Dongle 600 may include a secondary connector for interface when the dongle is not connected to a monitor. The secondary connector may be used for power or data transfer to or from the dongle.

In an embodiment an adapter is provided to interface with electrical connection port 206 and further interface with one or more of dongle 600. Using such an adapter the system may provide alternate configurations, such as may be desired for convenience or comfort.

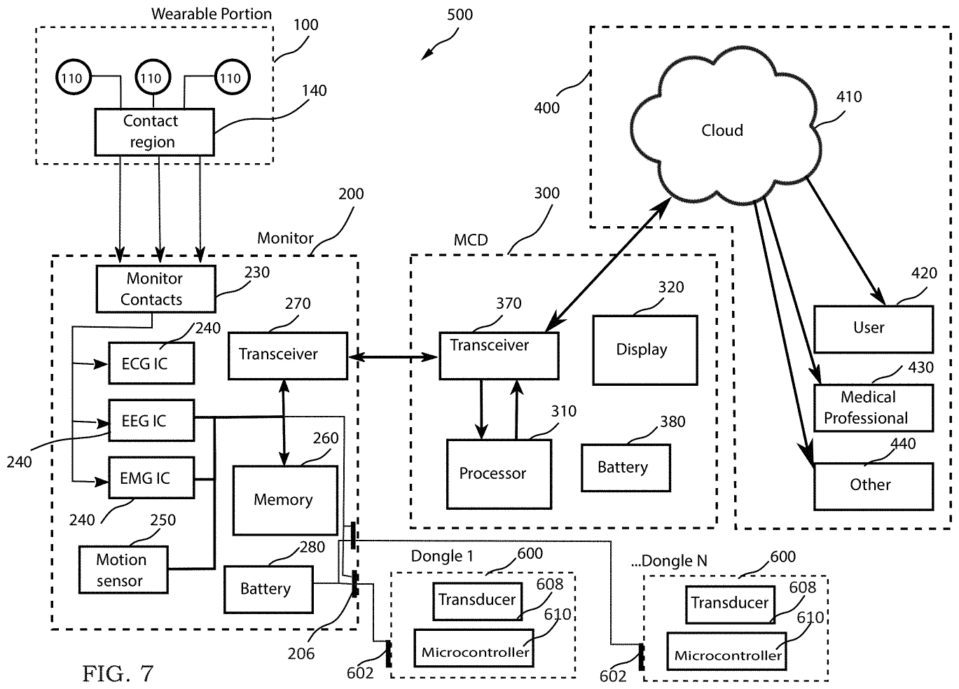

FIG. 7 is a schematic representation of electrophysiological monitoring system 500 including a wearable portion 100, monitor 200, and dongle 600 generally as described above. Electrophysiological monitor 200 includes at least one integrated circuit (IC) 240 configured to digitize biosignals received from sensors 110 which may be any one of ECG, EEG, or EMG signals. In the shown embodiment, monitor 200 includes three of IC 240, each of which digitizes one of ECG, EEG, or EMG signals. In another embodiment, a single IC 240 may be configured to digitize all of ECG, EEG, and EMG signals. Other IC configurations maybe readily envisioned to achieve the same result. IC 240 may further perform additional functions, such as signal amplification, filtering, lead-off detection, signal resampling, impedance measurement, etc. Signals processed by IC 240 in any of the above-mentioned manners are referred to herein as digitized signals.

One or more of dongle 600 are connected to monitor 200 by the interface of connector 602 with electrical connection port 206. Each dongle includes a transducer 608, which may be a sensor or an actuator. Transducer 608 may be coupled to dongle sensor contact 604 (see FIG. 6B). Transducer 608 is electrically coupled to a microcontroller 610 which receives signals from the transducer and may perform signal formatting, analysis, or signal feature detection. Microcontroller 610 is coupled to connector 602 and may transmit or receive data to or from monitor 200.

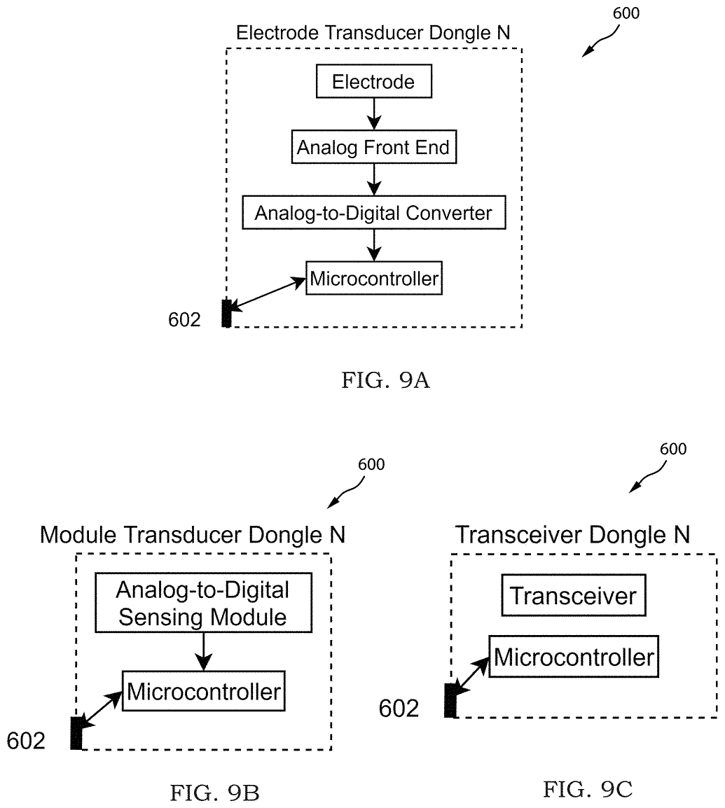

FIGS. 9A-9B are schematic representations of embodiments of dongle 600 including a transducer. In exemplary embodiments, transducer 608 may be used for acquiring one or more physiological parameters such as a temperature signal, a blood oxygen saturation signal, an analyte concentration (analytes including but not limited to glucose, sodium ions, potassium ions, chloride ions, ammonium ions, calcium ions, magnesium ions, bicarbonate, lactate, uric acid, ethanol), pH, bioimpedance, or a PPG signal. The previously mentioned signals provide information relating to, for example, sleep apnea, systolic or diastolic blood pressure, respiration rate, heart rate, cystic fibrosis, hypo- and hyperkalemia, acid-alkali balance disorders, kidney-stones, liver dysfunction, hyperparathyroidism, hypomagnesemia, cardiac arrhythmias, hypo- and hyperglycemic levels, lactic acidosis, heart failure, severe infections (sepsis), alcohol intoxication, euphoria, and other medical conditions. Possible applications for the above are found within prenatal care, neonatal care, pain management, oncology monitoring, blood monitoring, heart monitoring.

FIGS. 9C is a schematic representation of an embodiment of dongle 600 including a transceiver. The transceiver may be configured for wireless communication with the monitor, the MCD, or other dongles of the system. Other features provided for the dongle may include a magnetic feature, pairing features, and ability to interface with additional wearable monitoring devices. In embodiments, the dongle is configured for mechanical connection, electrical connection, or both, to at least one additional dongle.

Monitor 200 is powered by a replaceable or rechargeable battery 280, such as a standard button cell battery. Multiple batteries 280 may be provided with monitor 200 so that while a first battery is installed in monitor 200 a second battery may be recharged and ready to replace the first (in use) battery as needed. In this manner, monitor 200 may be used substantially uninterrupted for prolonged periods of time (up to several years). In some embodiments, connector 602 of the dongle is configured to receive power via interface with electrical connection port 206 of the monitor.

A transceiver 270 in monitor 200 wirelessly transmits digitized signals or motion sensor data to a mobile communications device (MCD) 300, such as a cellular telephone, tablet, or the like. In one embodiment, transceiver 270 uses the Bluetooth Low Energy (BLE) specification to prolong battery life of the monitor. In embodiments, a BLE transceiver may be left on continuously or may alternate between a full power “wake” mode and a lower power “sleep” mode. In other embodiments, transceiver 270 may use wireless internet communication, standard Bluetooth, or other communication protocols known in the art.

Monitor 200 includes a memory 260, such as a flash memory, ROM, EEPROM, or the like. In one embodiment, memory 260 is an SD card, and digitized signals may be stored internally to monitor 200 for up to a 24 hour period. In an embodiment, monitor 200 has two operating modes, Holter mode and monitor mode. When operated in Holter mode, digitized signals are stored on memory 260 of monitor 200 for a period of time such as 12, 24, 36, or 48 hours. When in Holter mode monitor 200 stores sensor data without transferring data to MCD 300 or other devices or networks. When monitor 200 is operated in monitor mode, digitized signals may be temporarily stored on memory 260 of monitor 200 and are transferred to MCD 300 either in pseudo-real time or as soon as a network connection is available.

MCD 300 includes a transceiver 370 for wirelessly receiving and transmitting signals to or from monitor 200 and a monitor network 400, which may be a cloud network or other internet network. While transceiver 370 is referred to herein in the singular, transceiver 370 may comprise multiple distinct hardware elements for communication via various protocols. For example, transceiver 370 may include a BLE transceiver for sending/receiving signals to/from monitor 200; a wireless network interface which supports a typical wireless local area network (WLAN), for example, Wi-Fi, or some other wireless local network capability, like, for example, femtocell or picocell wireless, Wireless USB, etc. for transmitting signals to monitor network 400; and/or an interface to a cellular network for transmitting signals to the monitor network.

Data transmitted from dongle 600 to monitor 200 may be stored in memory 260 or transmitted to transceiver 270 for communication to MCD 300.

In addition to transceiver 370 receiving digitized signals from monitor 200, transceiver 370 may transmit signals or instructions to monitor 200, such as to change the operational configuration of IC 240 (e.g., changing gain, sampling rate, or filter settings), to alternate between Holter and monitor modes, to query status of connections or battery levels, to request data transfer from memory 260, or other operational instructions.

MCD 300 further includes a processor 310 configured to process the digitized signals received from monitor 200 by transceiver 370. Processing performed by processor 310 may include signal filtering; artifact removal; comparison of digitized signals with databases of normal and pathologic ECG/EMM/EEG signals; template matching; detecting ECG abnormalities, such as arrhythmias and abnormalities in the morphology (“shape”) of the ECG wave which may be predictive of critical cardiac events; determining vital signs such as heart rate, respiration rate, physical activity index or blood pressure; detecting falls; and applying fast Fourier transform (FFT) to extract amplitudes or relevant frequencies for EMG and EEG signals. Outputs of any of the aforementioned processing performed on the MCD are referred to hereinafter as processed data.

MCD 300 further includes a display 320, which may display to the user certain digitized signals or processed data, and a battery 380. It is particularly advantageous to perform signal processing on processor 310 of MCD 300 rather than on monitor 200 itself, due to the high speed and processing power of commercially available MCDs at relatively low cost as compared to processors customized to specific applications. By minimizing the signal processing performed on-board monitor 200, the time before discharge of battery 280 may be extended and the overall size of the monitor reduced. Signal processing may be controlled via a mobile software application (app), suitable for installation on commercially available MCDs. In an embodiment, the MCD is dedicated for use with system 500.

Signals processed by processor 310 are transmitted by transceiver 370 to monitor network 400. In addition, unprocessed digitized signals received by MCD 300 may be transmitted to monitor network 400 for analysis or processing outside of MCD 300, such as by a medical professional connected to monitor network 400. When monitor 200 is in monitor mode and wireless communication channels are active, data transfer from MCD 300 to monitor network 400 is continuous.

In embodiments, monitor network 400 includes a cloud 410 which may be accessed by a number of clients such as a user 420 (the patient wearing device 100); a medical professional 430, which may be an individual or team of doctors, a hospital network, out-patient care provider, or similar; and other clients 440 such as an emergency points of contact, lay caregivers, patient supervisors, etc.

An interface module may enable client devices (computers, servers, mobile phones and the like) to manage connection to and view data received from the monitor network. The module may be installed locally on a client device or may be remotely hosted and accessed by the client device via an internet browser. The module may manage communication between devices within the monitor network, synchronize data between the MCD and client devices, and provide clients with a graphical user interface (dashboard') which may be customized for the type of client (user of device, medical professional, emergency contact, etc.). The module may enable multiple clients to access session data either in real-time or asynchronously.

FIG. 8 is an enlarged cross-sectional view along the line VIII-VIII of FIG. 3A, the view enlarged in height to better illustrate the thickness of the layers of the device. In the orientation shown, the bottommost layer is in contact with the skin of the patient during application and use.

In embodiments, conductive polymer pattern 102 comprises a high-conductivity polymer complex poly(3,4-ethylenedioxythiophene): poly(styrenesulfonate) (PEDOT:PSS). The polymer pattern may be ink-jet patterned onto a substrate sheet 104. Substrate sheet 104 may include three layers: i) a front liner 104a, made, for example, of paper; ii) a film 104b, which is flexible and skin-friendly, such as a polyurethane film, and iii) an adhesive layer 104c, including a biocompatible adhesive. Polymer pattern 102 is patterned on layer 104c of substrate sheet 104.

Wearable portion 100 is put in contact with the skin of a patient such that the front liner 104a is away from the body. Polymer pattern 102 contacts the skin of the patient, and is protected by film 104b which is adhered to the skin with adhesive layer 104c. Front layer 104a may be removed and discarded.

Before application of substrate sheet 104 to the skin, holes may be are cut in substrate sheet 104 corresponding to the locations of additional sensors of the dongle.

In embodiments, conductive polymer pattern 102 and layers 104b and 104c of the substrate sheet have a combined thickness of less than 1 micrometer, less than 750 nanometers (nm), less than 700 nm, less than 650 nm, or less than 600 nm. In an example embodiment, conductive polymer pattern 102 has a thickness of about 250 nm.

In an embodiment, ultrathin wires connect each terminus 130 to a sensor contact 150. In another embodiment, the wires are sandwiched between two layers of tape which provide support and protection for the wires. In embodiments, one end of each wire is located between a terminus 130 of conductive polymer pattern 102 and film 104b. The other end of each wire is connected to a sensor contact 150, such as by clipping between a male and female component of sensor contact 150.

In another embodiment of the electrical connection between terminus 130 and sensor contact 150, the connection may include a substantially planar layer of conductive polymer, such as PEDOT:PSS, rather than ultrathin wires. In embodiments, the conductive polymer may be printed on a support layer to which sensor contacts 150 are connected.

In an embodiment, contact region 140 comprises a nonconductive support layer 160 to which the sensor contacts are connected. For example support layer 160 may be made from polyethylene terephthalate (PET), and sensor contacts 150 may be electroconductive studs having front and back components which are connected with the PET support layer between the front and back components. In an embodiment, contact region 140 may not be attached to the skin of the patient and may be supported by connection to the film 104b.

In an embodiment, monitor 200 includes a motion sensor 250 such as an accelerometer or piezoelectric sensor, for detecting sudden movements of the patient, such as a fall. Monitor 200 may also monitor other biosignals such heart rate or respiration rate.

Referring again to FIG. 1,an embodiment of the monitor is shown with a connected dongle. In embodiments, the dongle has a dongle thickness which is less than a sidewall thickness of the monitor. In one example, the dongle has a dongle thickness of about 11 mm. In an embodiment, the dongle is sized to be supported by the monitor through the interface of the connector with the electrical connection port. In such an embodiment the dongle is not further attached to or supported on the body of the patient. In another embodiment the dongle may be additionally supported with an adhesive backing.

The embodiments of the system described herein are exemplary and numerous modifications, combinations, variations, and rearrangements can be readily envisioned to achieve an equivalent result, all of which are intended to be embraced within the scope of the appended claims. Further, nothing in the above-provided discussions of the system should be construed as limiting the invention to a particular embodiment or combination of embodiments. The scope of the invention is defined by the appended claims.

We claim:

1. A multifunctional electrophysiological monitoring system, comprising:
a conductive polymer pattern configured for transfer to the skin of a patient, the pattern including a plurality of sensor regions each connected to a patterned lead having a terminus adjacent to a common contact region;
a plurality of sensor contacts arranged within the contact region, each terminus of the patterned leads in electrical communication with one of the sensor contacts;
an electrophysiological monitor having:
a housing having a rear face and a sidewall;
a plurality of monitor contacts on the rear face, each of the monitor contacts configured to directly connect to one of the sensor contacts;
an integrated circuit configured to digitize a first biosignal;
a memory;
a transceiver configured for wireless transmission of the digitized signals; and,
an electrical connection port in the sidewall;
a dongle configured to perform a supplemental function and having a connector configured to interface with the electrical connection port to communicate information to the monitor; and
a mobile communication device having a transceiver configured for wireless communication with the monitor and a processor configured to process the digitized signals.

2. The system of claim 1, wherein the supplemental function includes acquiring information about a physiological parameter, the acquired information different from the first biosignal sensed by the monitor.

3. The system of claim 1, wherein the information acquired by the dongle is selected from the group consisting of: a temperature signal, a blood oxygen saturation signal, an analyte concentration, a pH measurement, a bioimpedance measurement, or a photoplethysmogram signal.

4. The system of claim 1, wherein the first biosignal is selected from the group consisting of:
an ECG signal, an EEG signal or an EMG signal.

5. The system of claim 1, wherein the monitor includes a plurality of electrical connection ports in the sidewall.

6. The system of claim 5, further including an additional dongle configured to acquire information regarding an additional biosignal and having a connector configured to interface with one of the electrical connection ports to communicate the information to the monitor.

7. The system of claim 6, wherein the additional biosignal relates to a different physiological property than the first biosignal.

8. The system of claim 1, wherein the connector of the dongle is configured to interface with the electrical connection port to receive power.

9. The system of claim 1, wherein the dongle has a dongle thickness of less than a sidewall thickness of the monitor.

10. The system of claim 1, wherein the dongle is sized to be supported by the monitor through the interface of the connector with the electrical connection port.

11. The system of claim 1, wherein each of the monitor contacts is configured to directly connect both mechanically and electrically to one of the sensor contacts.

12. The system of claim 1, wherein the dongle includes a microcontroller electrically coupled to the connector and a transducer electrically coupled to the microcontroller.

13. The system of claim 1, wherein the dongle includes a transducer electrically coupled to a dongle sensor contact configured to contact the skin of the patient.

14. The system of claim 1, wherein the supplemental function of the dongle includes acquiring information about a physiological parameter through electrical communication with an electrode.

15. The system of claim 1, wherein the dongle includes a sensing module to acquire information about a physiological parameter.

16. The system of claim 1, wherein the dongle includes a transceiver configured for wireless communication with the mobile communication device.

17. The system of claim 1, wherein the dongle is configured for mechanical and electrical to at least one additional dongle.
